(19) United States
(12) Patent Application Publication
(10) Pub. No.: US 2023/0032150 A1
(43) Pub. Date: Feb. 2, 2023

(54) DRILL BIT AND SUBCORTICAL EXPANDING BONE ANCHOR FOR TENODESIS

(71) Applicant: Conmed Corporation, Largo, FL (US)

(72) Inventors: Andrew Kam, Odessa, FL (US); Peter Verdonk, Zwijnaarde (BE)

(73) Assignee: Conmed Corporation, Largo, FL (US)

(21) Appl. No.: 17/789,664

(22) PCT Filed: Jan. 4, 2021
(86) PCT No.: PCT/US21/12075

§ 371 Date: Jun. 28, 2022

Related U.S. Application Data
(60) Provisional application No. 62/956,880, filed on Jan. 3, 2020.
(60) Provisional application No. 62/984,828, filed on Mar. 4, 2020.

(51) Int. Cl. A61B 17/16; A61F 2/08; A61B 17/04

(52) U.S. Cl. CPC A61B 17/1615; A61F 2/0811; A61B 17/0401; A61F 2002/0888; A61B 2017/0412; A61B 2017/0414; A61B 2017/0464; A61F 2002/0852

(57) ABSTRACT

A drill bit for bone surface preparation and creation of a shallow pilot hole and a bone anchor. The drill bit includes a shaft extending to a distal end, the distal end having a drill tip, a sharp point extending distally from the drill tip, and a stop extending around the shaft at the drill tip, the stop having one or more cutting edges and a diameter that is greater than a diameter of the shaft. The bone anchor includes a body having at least two opposing openings forming a window extending through the body, an expansion member having a pair of connected and spaced wings, the expansion member extending at least partially into the window in the body, and a fixation mechanism comprising an adjustable loop, the adjustable loop extending between the pair of connected and spaced wings in the window of the body.

### CROSS-REFERENCE TO RELATED APPLICATION

The present application claims priority to and the benefit of U.S. Provisional Patent Application No. 62/956,880, filed on Jan. 3, 2020 and entitled “Subcortical Expanding Bone Anchor for Tenodesis” and U.S. Provisional Patent Application No. 62/984,828, filed on Mar. 4, 2020 and entitled “Drill Bit with Bone Surface Preparation,” the entireties of which are incorporated herein by reference.

### BACKGROUND OF THE INVENTION

### 1. Field of the Invention

The present invention relates to a drill bit and bone anchor and, more particularly, to a drill bit with cutting edges for preparing a bone surface and drilling a pilot hole for a bone anchor and an expanding bone anchor.

### 2. Description of Related Art

Current devices used for tenodesis, specifically Lateral Extra-articular Tenodesis (LET), include staples, interference screws, and radially expanding anchor devices. Staples are bulky and large, which may lead to post-operative irritation that would require removal after the healing period. Interference screws require rotation of the implant along the graft for insertion, which could lead to wrapping of the graft, leading to undesirable orientation and/or tensioning of the graft. In addition, the use of interference screws typically requires the user to drill a tunnel through the bone to be able to shuttle the graft into the bone socket. Radial expansion devices are long (>10 mm) which require the length of graft within the tunnel to be just as long. Harvesting of a longer graft may require a longer incision.

During a soft tissue repair, in which soft tissue is reattached to bone, it is typical to prepare the surface of the bone by removing the periosteum to create a healing bed around the anchor point. Currently, this is achieved by trimming the soft tissue with scissors and then removing the periosteum with a blade or rasp.

Therefore, there exists a need for a bone anchor and a method for bone surface preparation and creation of a shallow pilot hole for the bone anchor.

The term “suture” as used herein may be any type of filamentous material such as a biocompatible or bioabsorbable filament, ribbon, tape, woven or non-woven material.

Description of the Related Art Section Disclaimer: To the extent that specific patents/publications/products are discussed above in this Description of the Related Art Section or elsewhere in this disclosure, these discussions should not be taken as an admission that the discussed patents/publications/products are prior art for patent law purposes. For example, some or all of the discussed patents/publications/products may not be sufficiently early in time, may not reflect subject matter developed early enough in time and/or may not be sufficiently enabling so as to amount to prior art for patent law purposes. To the extent that specific patents/publications/products are discussed above in this Description of the Related Art Section and/or throughout the application, the descriptions/disclosures of which are all hereby incorporated by reference into this document in their respective entirety(ies).

### BRIEF SUMMARY OF THE INVENTION

Embodiments of the present invention are directed to a drill bit for bone surface preparation and creation of a shallow pilot hole. An embodiment of the drill bit includes a shaft extending to a distal end, the distal end having a drill tip, a sharp point extending distally from the drill tip, and a stop extending around the shaft at the drill tip, the stop having one or more cutting edges and a diameter that is greater than a diameter of the shaft.

According to another aspect, the present invention is a bone anchor. The bone anchor includes a body having at least two opposing openings forming a window extending through the body, an expansion member having a pair of connected and spaced wings, the expansion member configured to extend at least partially into the window in the body, and a fixation mechanism comprising an adjustable loop, the adjustable loop extending between the pair of connected and spaced wings in the window of the body. The adjustable loop can further extend through a first aperture through the top surface of the body, through a first aperture at the bottom of the expansion member, along the bottom surface of the expansion member, back though a second aperture through the bottom surface of the expansion member, and out through the a second aperture through the top surface of the body. The adjustable loop is configured to move the expansion member up into the body (per a force on the proximal end of the adjustable loop in the relative up direction) when tissue is positioned through the window until wings pop through respective side holes in the body and expand outward to lock the assembly in a deployed configuration.

These and other aspects of the invention will be apparent from and elucidated with reference to the embodiment(s) described hereinafter.

### BRIEF DESCRIPTION OF THE SEVERAL VIEWS OF THE DRAWING(S)

The present invention will be more fully understood and appreciated by reading the following Detailed Description in conjunction with the accompanying drawings. The accompanying drawings illustrate only typical embodiments of the disclosed subject matter and are therefore not to be considered limiting of its scope, for the disclosed subject matter may admit to other equally effective embodiments. Reference is now made briefly to the accompanying drawings, in which:

FIG. 1A is a close-up, perspective view of a distal end of a drill bit, according to an embodiment;

FIG. 1B is a bottom, perspective view of the distal end of the drill bit, according to an embodiment;

FIG. 1C is a bottom view of the distal end of the drill bit, according to an embodiment;

FIG. 2 is a perspective view of a bone anchor, according to an embodiment;

FIG. 3 is a perspective view of a body of the bone anchor, according to an embodiment;

FIG. 4 is a perspective view of an expansion member of the bone anchor, according to an embodiment;

FIG. 5 is a perspective view of a fixation mechanism of the bone anchor, according to an embodiment;

FIG. 6 is a side, perspective view of a driver loaded with the body and expansion member of the bone anchor, according to an embodiment;

FIG. 7 is an exploded view of the driver of FIG. 6;

FIG. 8 is a bottom view of the driver of FIG. 6;

FIG. 9 is a top view of the bone anchor loaded on the driver in the pre-deployment configuration, according to an embodiment;

FIG. 10 is a close-up, top view of the bone anchor and driver of FIG. 9;

FIG. 11 is a top view of the bone anchor loaded on the driver in the post-deployment configuration, according to an embodiment; and

FIG. 12 is a close-up, top view of the bone anchor and driver of FIG. 11.

### DETAILED DESCRIPTION OF THE INVENTION

Aspects of the present invention and certain features, advantages, and details thereof, are explained more fully below with reference to the non-limiting examples illustrated in the accompanying drawings. Descriptions of well-known structures are omitted so as not to unnecessarily obscure the invention in detail. It should be understood, however, that the detailed description and the specific non-limiting examples, while indicating aspects of the invention, are given by way of illustration only, and are not by way of limitation. Various substitutions, modifications, additions, and/or arrangements, within the spirit and/or scope of the underlying inventive concepts will be apparent to those skilled in the art from this disclosure.

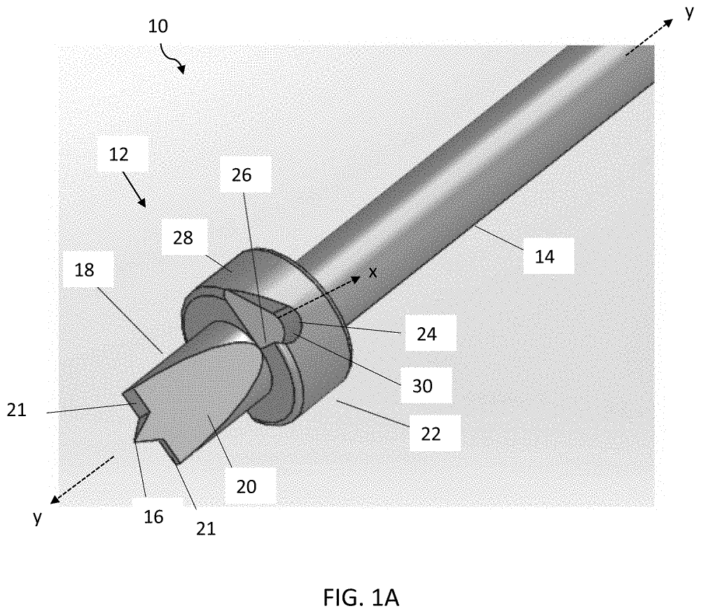

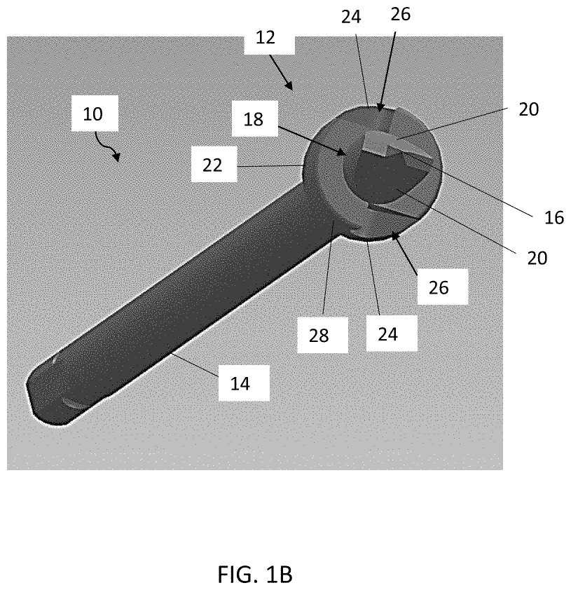

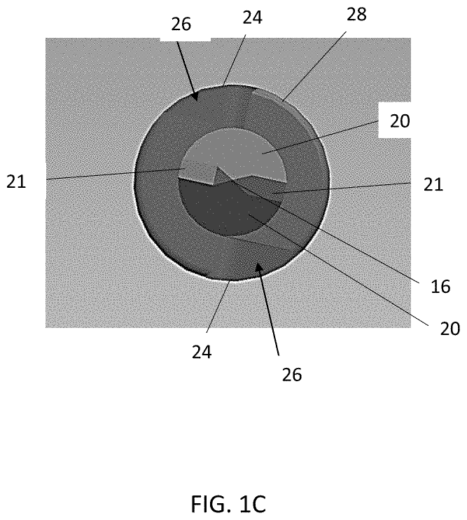

Referring now to the figures, wherein like reference numerals refer to like parts throughout, FIG. 1A shows a close-up, perspective view of a distal end 12 of a drill bit 10, according to an embodiment. The drill bit 10 in FIG. 1A comprises a shaft 14 extending to a distal end 12. The distal end 12 comprises features suitable for creating a pilot hole in bone for insertion of a bone anchor. Specifically, the distal end 12 of the drill bit 10 terminates in a sharp point 16. In the depicted embodiment, the sharp point 16 is substantially triangular and is tapered to be larger in width in the proximal direction. Alternatively, there can be no points, or the drill tip 18 itself can be tapered to a point. There can also be multiple points of the same or different sizes and/or multiple points of the same or different shapes, which can extend from the most distal surface of the tip and/or the sides of the tip.

Still referring to FIG. 1A, the sharp point 16 extends from a drill tip 18. The drill tip 18 is substantially cylindrical with one or more flat faces 20. According to an embodiment, there are two flat faces 20, as shown in FIG. 1B. The two flat faces 20 oppose each other and converge at the sharp point 16, as shown in FIGS. 1A-1C. At least a portion of the sharp point 16 is substantially flush or planar with the flat faces 20 and at least a portion of the two flat faces 20 are connected via substantially flat or planar distal surface 21. In FIGS. 1A and 1C, there are two distal surfaces 21 with the sharp point 16 extending between them.

The drill bit 10 further comprises a stop 22 at the distal end 12 of the shaft 14. As shown in FIG. 1A, the stop 22 is a cylindrical collar that is proximal relative to the drill tip 18. The stop 22 comprises a diameter that is greater than a diameter of the shaft 14 and the drill tip 18 (including the sharp point 16) of the drill bit 10. The larger diameter of the stop 22 allows the stop 22 to function as a depth limiting stop. In other words, the stop 22 prevents the drill bit 10 from creating a pilot hole that is too deep.

In the depicted embodiment, the stop 22 comprises one or more cutting edges 24. In FIGS. 1B-1C, the stop 22 comprises two cutting edges 24. The cutting edges 24 are created in the stop 22 by recessing the stop 22. In FIGS. 1A-1C, recesses 26 are formed through an outer surface 28 of the stop 22, each recess 26 creating a cutting edge 24. The outer surface 28 of the stop 22 is the bone contacting surface. As shown in FIGS. 1B-1C, the two cutting edges 24 are on opposing sides of the flat faces 20 and sharp point 16.

In FIG. 1A, the shaft 14 extends along a central longitudinal y-y axis. An inner surface 30 of the stop 22 extends from the recess 26 to the cutting edges 24 along an axis x-x. In the embodiment shown, the axis x-x is substantially perpendicular to the central longitudinal y-y axis. The cutting edges 24 skim the surface of the bone to prepare a healing bed without aggressively cutting into the bone. The outer surface 28 of the stop 22 in FIGS. 1A-1C is smooth; however, according to other embodiments, the outer surface 28 does not have cutting edges 24 but is abrasive.

In use, during reattachment of soft tissue to bone procedures, such as tenodesis, the bone surface must be prepared to create a healing bed. The healing bed is created by engaging the cutting edges 24 (or abrasive outer surface 28) of the drill bit 10 with the bone surface. After the healing bed is created, the pilot hole is created for a bone anchor. The pilot hole is created by drilling into the bone surface with the drill tip 18 (and sharp point 16) until the stop 22 is reached. Once the pilot hole is created, a bone anchor can be inserted and deployed. The drill bit 10 can also have attachment options for the drill tip 18, including ¼ inch Jacobs and Trinkle attachments.

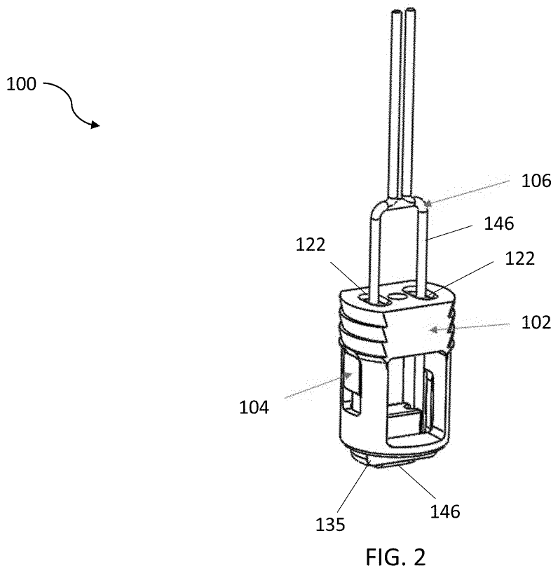

Turning now to FIG. 2, there is shown a perspective view of a bone anchor 100, according to an embodiment. The bone anchor 100 is sized and configured for fixation within a shallow pilot hole (e.g., 10 mm or less), such as one created by the drill bit 10 described above with reference to FIGS. 1A-1C. The bone anchor 100 comprises a body 102 with an expansion member 104 and a fixation mechanism 106. The bone anchor 100 described below and shown in FIGS. 2-5 preferably does not require rotation for insertion and deployment.

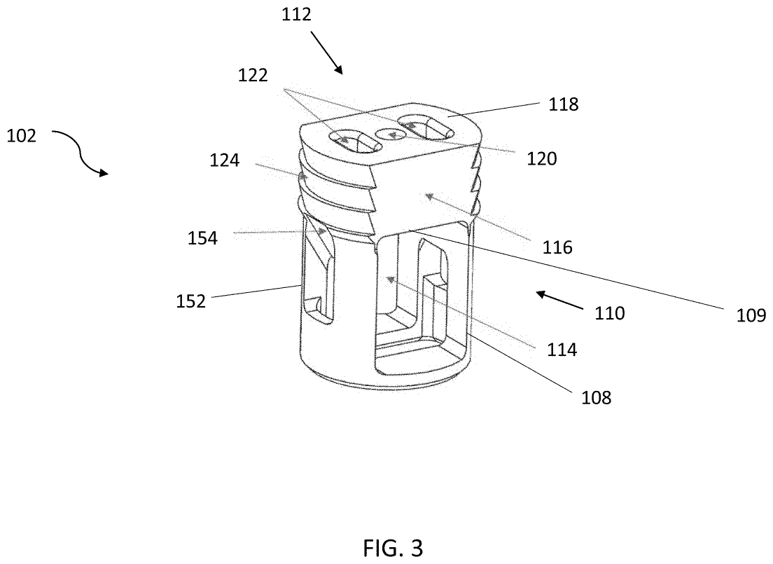

Referring now to FIG. 3, there is shown a perspective view of the body 102 of the bone anchor 100, according to an embodiment. The body 102 of the bone anchor 100 is substantially cylindrical or rectangular. The body 102 comprises two opposing openings 108 (on opposing sides 110, 112 of the body 102) that form a window 114 extending through the body 102. In the depicted embodiment, the two opposing openings 108 are rectangular; however, they can be any size and shape suitable for maintaining a graft (not shown) in/through the window 114.

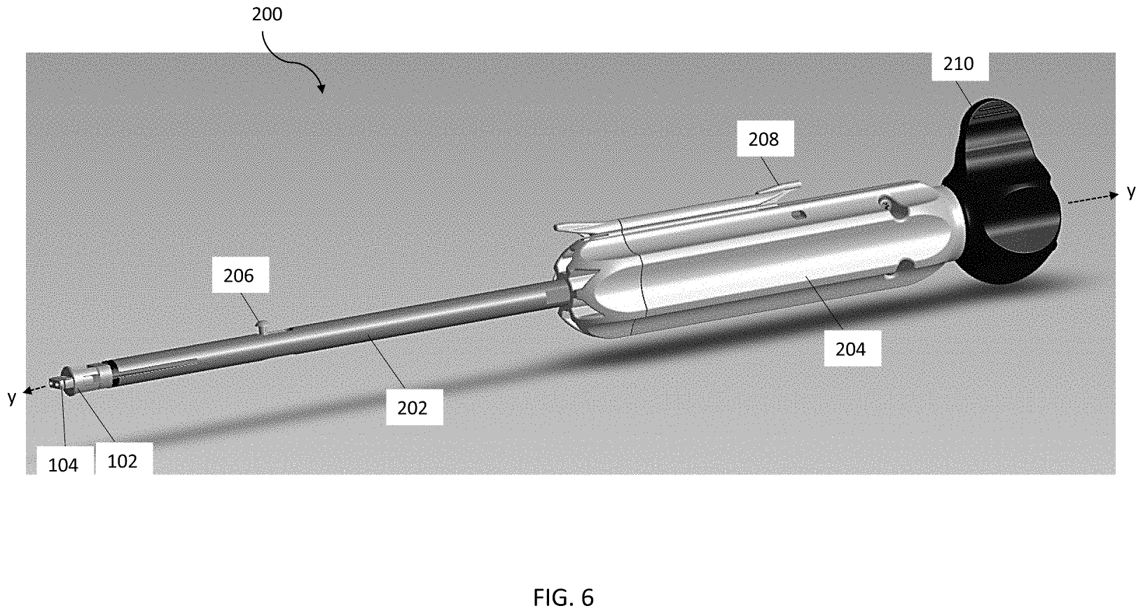

As shown in FIG. 3, the body 102 comprises two opposing sidewalls 116 (on opposing sides 110, 112 of the body 102). The sidewalls 116 are substantially flat or planar surfaces that extend from the openings 108 to a top surface 118 of the body 102. The flat sidewalls 116 allow the graft (not shown) to be compressed against a bone tunnel wall. The top surface 118 of the body 102 includes an interface 120 for an anchor driver or inserter (not shown). The interface 120 fits any number of handheld drill motor portions. In the depicted embodiment, the interface 120 is an aperture, a round or circular aperture, extending at least partially through the top surface 118 for interfacing with an anchor driver or inserter 200 (FIGS. 6-12).

Still referring to FIG. 3, the top surface 118 of the body 102 additionally includes one or more openings 122 sized and configured to receive the fixation mechanism 106 therethrough. In the depicted embodiment, the one or more openings 122 include two oblong openings 122. The interface 120 is between the two oblong openings 122 in FIG. 3. The two oblong openings 122 allow for the fixation mechanism 106 to couple with the expansion member 104. As also shown in FIG. 3, the body 102 comprises an abrasive feature, such as ridges 124. In the depicted embodiment, the ridges 124 extend between the sidewalls 116 of the body 102. The purpose of the ridges 124 is to engage with the cortex of the bone.

As shown in FIG. 3, the body 102 additionally comprises one or more opposing apertures 152 (note, only one aperture 152 is shown in FIG. 3). In the depicted embodiment, the apertures 152 are substantially rectangular or oblong. In the embodiment shown in FIG. 2, the apertures 152 are smaller than the openings 108 that create the window 114 in the body 102. As also shown in FIG. 3, the body 102 has one or more interior ramps 154 connected to the apertures 152. The purpose of the apertures 152 and ramps 154 are sized and configured to receive components of the expansion member 104, as described in detail below.

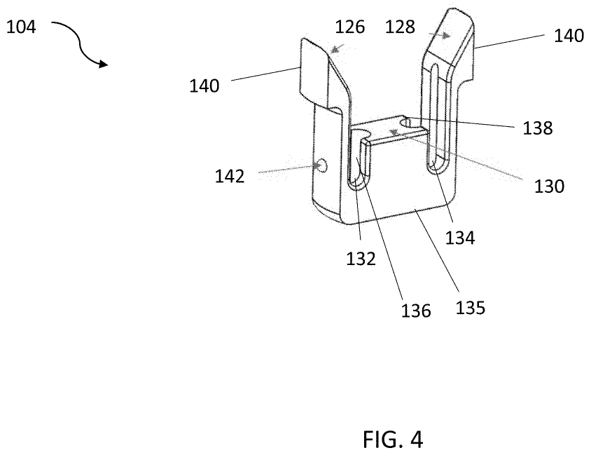

Turning now to FIG. 4, there is shown a perspective view of the expansion member 104 of the bone anchor 100, according to an embodiment. The expansion member 104 in FIG. 4 comprises a first wing 126 connected to a second wing 128. In the depicted embodiment, the first and second wings 126, 128 are arms that are spaced and connected in a U-shape such that the first and second wings 126, 128 and substantially parallel.

Still referring to FIG. 4, the expansion member 104 include a projection 130 extending between the first and second wings 126, 128. The purpose of the projection 130 is to compress a graft (not shown) within the window 114 of the body 102. As shown, the projection 130 extends substantially parallel to the first and second wings 126, 128. In the depicted embodiment, a first channel 132 extends between the first wing 126 and a first side 136 of the projection 130 and a second channel 134 extends between the second wing 128 and a second side 138 of the projection 130. The first and second channels 132, 134 are semi-circular through the first and second sides 136, 138 of the projection 130, respectively.

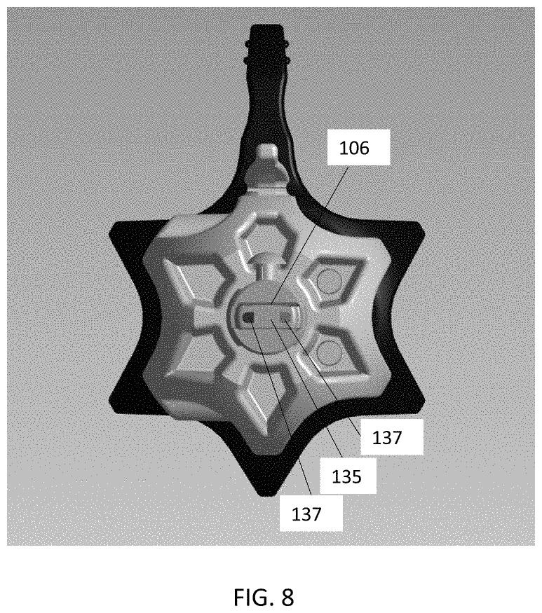

As shown in FIG. 4, the first wing 126, second wing 128, and projection 130 are connected by a distal end 135 of the expansion member 104. The distal end 135 has two apertures 137 extending therethrough. The apertures 137 are shown clearly in FIG. 8. One aperture 137 extends to the first channel 132 and the other aperture 137 extends to the second channel 134. In a pre-deployment configuration of the bone anchor 100, as shown in FIG. 2, the distal end 135 of the expansion member 104 extends distally out from the body 102 of the bone anchor 100.

As also shown in FIG. 4, the first and second wings 126, 128 are substantially straight with a protruding tip 140. The tip 140 of the first wing 126 extends in an opposite direction as the tip 140 of the second wing 128. Each tip 140 has both a rectangular cross-section and a substantially triangular cross-section. The first and second wings 126, 128 are at least semi-flexible such that the tips 140 of the first and second wings 126, 128 can be compressed outward, i.e., away from each other. When the bone anchor 100 is deployed, the tips 140 extend into the apertures 152 in the body 102 against the interior ramps 154 and expand outward and subcortically for fixation.

Further, at least one of the first and second wings 126, 128 can comprise a locking mechanism 142. In the embodiment shown in FIG. 4, the locking mechanism 142 is a round ball extending from the first wing 126. The second wing 128 can have a similar ball as well. The ball 142 is sized and configured to fit within a catch (not shown) on the body 102 so that the expansion member 104 can be locked into the body 102 when the bone anchor 100 is deployed (i.e., in a post-deployment configuration).

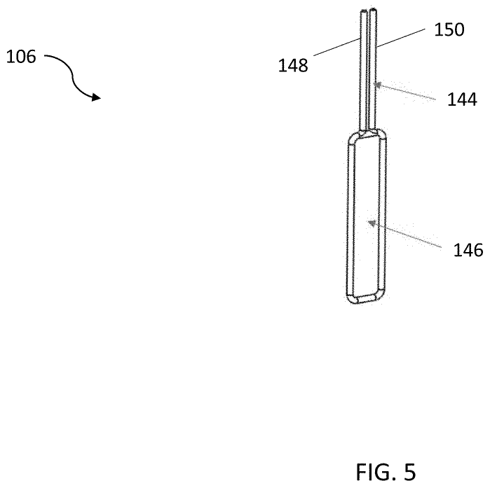

Referring now to FIG. 5, there is shown a perspective view of the fixation mechanism 106 of the bone anchor 100, according to an embodiment. The fixation mechanism 106 is preferably comprised of suture, but it can be composed of any suitable surgical material. The fixation mechanism 106 in FIG. 5 is a length of suture 144 comprising a loop 146. The loop 146 is an adjustable loop that secures the graft (not shown) to the bone anchor 100. The loop 146 is sized and configured to fit within the first and second channels 132, 134 of the expansion member 104, as shown in FIG. 2.

Specifically, the loop 146 extends in the first and second channels 132, 134 of the expansion member 104 and through and between the two apertures 137 (FIG. 8) in the distal end 135 of the expansion member 104. Therefore, the loop 146 secures the distal end 135 of the expansion member 104, extends through the first and second channels 132, 134 and then through the two oblong openings 122 in the top surface 118 of the body 102, as shown in FIG. 2. Thus, the fixation mechanism 106, particularly the loop 146, couples the expansion member 104 to the body 102 while maintaining an opening in the loop 146 for the graft (not shown) to be loaded within the window 114 of the body 102. The loop 146 also functions to actuate the expansion member 104 and hold the relative position of the expansion member 104 to the body 102 in a pre-deployment and post-deployment configurations, as described in detail below.

Turning back to FIG. 5, the first limb 148 of the length of suture 144 extends from the loop 146 and a second limb 150 of the length of suture 144 extends from the loop 146. In the depicted embodiment, the first and second limbs 148, 150 are adjacent and extend in the same direction, proximally, from the loop 146. In particular, the first and second limbs 148, 150 extend proximally from the loop 146 outside the body 102 of the bone anchor 100. The first and second limbs 148, 150 secure the graft (not shown) to the bone anchor 100.

Referring now to FIGS. 6-12, there are shown various views of a driver 200 loaded with some or all components of the bone anchor 100 in pre-deployment and post-deployment configurations. FIG. 6 is a side, perspective view of the driver 200 loaded with the body 102 and expansion member 104 of the bone anchor 100, according to an embodiment. The driver 200 comprises a driving shaft 202 connected to and extending to a handle 204. The driving shaft 202 comprises a tensioning mechanism 206 and the handle 204 comprises a locking mechanism 208. In the embodiment shown in FIG. 6, the handle 204 is further connected to a rotation mechanism 210.

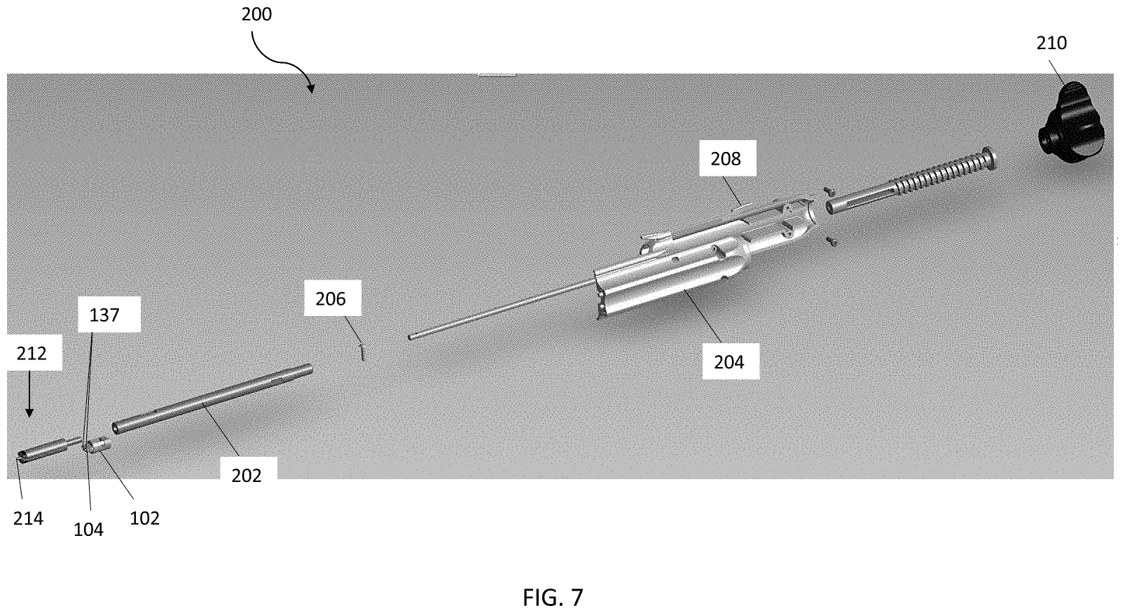

Turning now to FIG. 7, there is shown an exploded view of the driver 200 of FIG. 6. As shown, the rotation mechanism 210 of the driver 200 is a knob. The knob 210 connects to the tensioning mechanism 206. According to an embodiment, the tensioning mechanism 206 is a pin 206 that is connected to the knob 210 and extends through the driving shaft 202 (FIG. 6). Rotation of the knob 210 causes axial movement of the pin 206 along a central longitudinal axis y-y (FIG. 6) extending through the driver 200. In other words, rotation of the knob 210 causes the pin 206 to move proximally or distally along the driving shaft 202. As also shown in FIG. 7, the driver 200 comprises a distal end 212 having an interface 214 for engaging the interface 120 (FIG. 3) of the body 102 of the bone anchor 100. When the bone anchor 100 is loaded onto the driver 200, the expansion member 104 is the distalmost feature with its apertures 137 (FIG. 8) extending along the central longitudinal y-y axis, as shown in FIGS. 6-8.

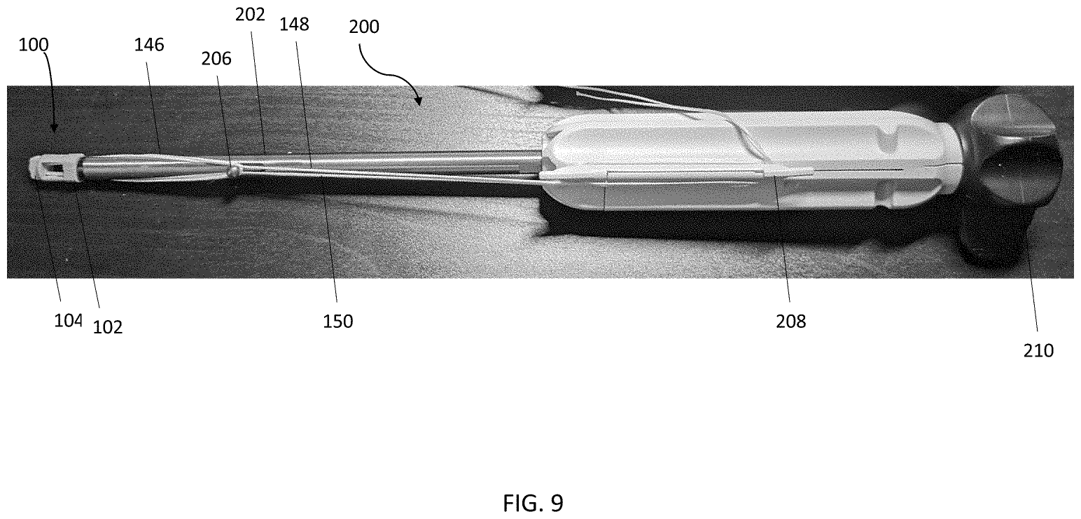

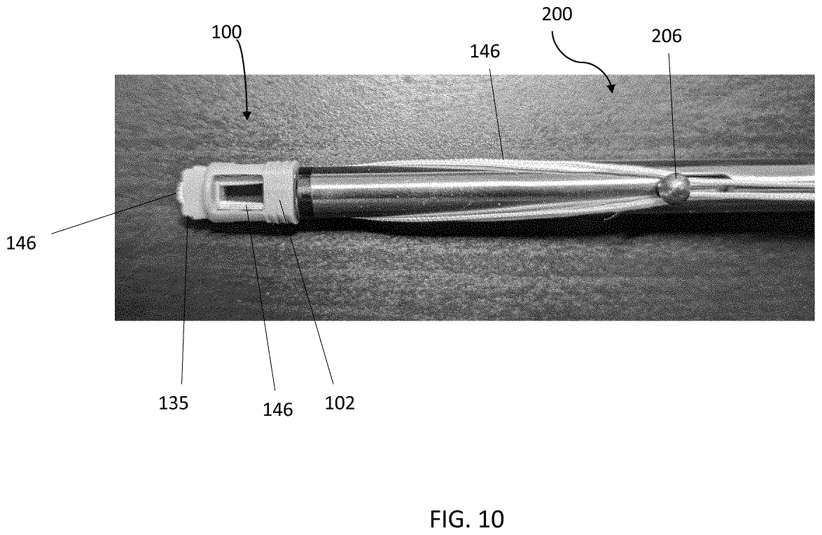

Referring now to FIGS. 9 and 10, there are shown top and close-up top views, respectively, of the bone anchor 100 loaded on the driver 200 in the pre-deployment configuration, according to an embodiment. When the bone anchor 100 is loaded on the driver 200 the loop 146 of the expansion member 104 extends between and through the apertures 137 in the distal end 135 of the expansion member 104. The loop 146 extends from the apertures 137 through the first and second channels 132, 134 between the first and second wings 126, 128 and out of the openings 122 through the top surface 118 of the body 102. From the openings 122 in the top surface 118 of the body 102, the loop 146 is wrapped around the tensioning mechanism 206 (i.e., the pin 206). The pin 206 holds the bone anchor 100 in the pre-deployment configuration shown in FIGS. 9 and 10.

Still referring to FIGS. 9 and 10, the first and second limbs 148, 150 extending from the loop 146 to the locking mechanism 208. In FIGS. 6-12, the locking mechanism 208 is a cleat on the handle 204 of the driver 200. In use, the first and second limbs 148, 150 extending from the loop 146 are wrapped around the cleat 208 to lock in or otherwise maintain the tension on the loop 146 in the pre-deployment configuration. As also shown in FIG. 9, the rotation mechanism 210 (i.e., knob 210) is in a first position when the driver 200 is in the pre-deployment configuration.

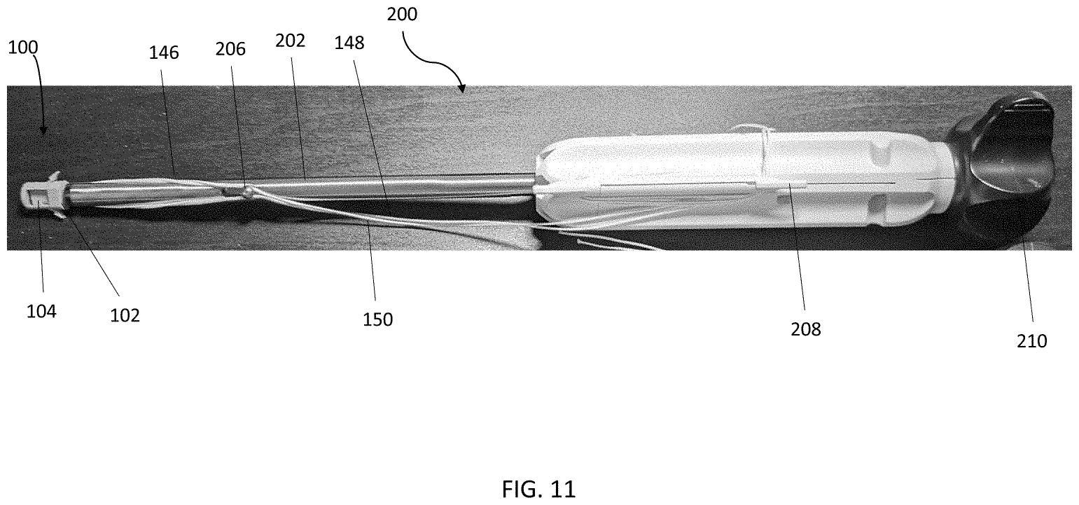

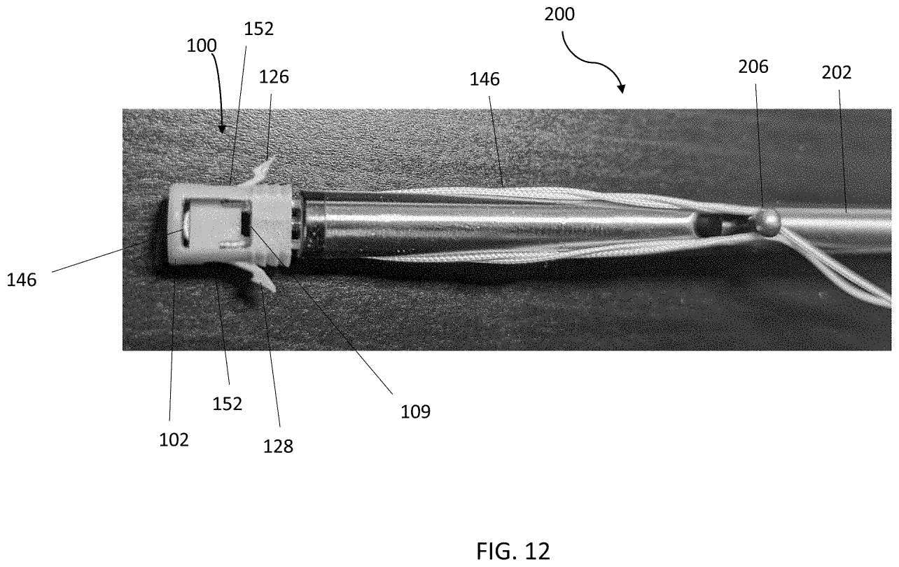

Referring now to FIGS. 11 and 12, there are shown top and close-up top views, respectively, of the bone anchor 100 loaded on the driver 200 in the post-deployment configuration, according to an embodiment. To move the driver 200 from the pre-deployment configuration to the post-deployment configuration, the rotation mechanism 210 (i.e., knob 210) is rotated from a first position (FIG. 9) to a second position (FIG. 11). When the knob 210 is rotated to the second position, the tensioning mechanism 206 (i.e., pin 206) is moved proximally along the driving shaft 202, as shown in FIGS. 11 and 12.

Because the tension in the loop 146 was locked via the locking mechanism 208 (i.e., cleat 208) in the pre-deployment configuration, proximal movement of the pin 206 causes the expansion member 104 to be pulled proximally into the body 102 of the bone anchor 100, as shown in FIG. 12. In the post-deployment configuration, the distal end 135 of the expansion member 104 is within the body 102 of the bone anchor 100. When the loop 146 is pulled by the pin 206 into the post-deployment configuration, the loop 146 pulls the first and second wings 126, 128 of the expansion member 104 proximally, forcing the first and second wings 126, 128 to slide along the ramps 154 (FIG. 3) in the body 102 and out through the apertures 152 in the body 102, as shown in FIG. 12.

In use, a pilot hole is drilled into the bone (using a drill bit 10 such as that shown in FIG. 1, for example). The pilot hole is sized to accommodate the bone anchor 100. To use the bone anchor 100, first, a free end of a harvested graft (not shown), such as a soft tissue graft, is inserted through the openings 108 and into the window 114 of the body 102 of the bone anchor 100 while the bone anchor 100 is in the pre-deployment configuration (FIGS. 9-10). The bone anchor 100 is then placed into the pilot hole. The bone anchor 100 can be inserted by engaging the interface 120 of the bone anchor 100 with the anchor driver 200 (or other inserter).

The driver 200 that is interfacing with the bone anchor 100 can be impacted with a mallet until a positive stop is reached, signaling that appropriate bone anchor 100 insertion depth has been reached. The position of the bone anchor 100 is maintained while the knob 210 on the driver 200 is rotated, which causes axial tension to be placed on the fixation mechanism 106 (i.e., loop 145). This draws the expansion member 104 into the body 102 (axial movement of the expansion member 104 with respect to the body 102), as shown in FIGS. 11-12. As a result of the interactions with the body 102, the first and second wings 126, 128 of the expansion member 104 will extend outwardly while the projection 130 compresses the graft (not shown) against the upper inside wall 109 (FIGS. 3 and 12) of the window 114.

Thereafter, the driver 200 can be removed from the bone anchor 100 and the user can secure the graft and bone anchor 100 with the first and second limbs 148, 150 of the length of suture 144. The first and second limbs 148, 150 (attached to the graft) can be loaded within the loop 146. Once loaded, the first and second limbs 148, 150 of the adjustable loop 146 are pulled in tension causing the loop 146 to constrict around the graft, holding the graft to the bone anchor 100. The first and second limbs 148, 150 may now be used to tie surgical knots to secure the loop 146, thereby securing the graft and the bone anchor 100 in place.

According to an embodiment, the bone anchor 100 can be a shallow anchor for knee procedures. The shallow depth of the bone anchor 100 avoids potential tunnel convergence with an ACL tunnel. The bone anchor 100 can also be used for lateral extraarticular tenodesis. It accommodates an approximately 10 mm wide×1.5 mm thick graft (e.g., strip of harvested IT band). The bone anchor 100 can be a single-use delivery device. The body 102 and the expansion member 104 of the bone anchor 100 can be composed of non-absorbable PEEK. The fixation mechanism 106 can be a length of suture 144 composed of UHMWPE suture. The bone anchor 100 can provide sufficient fixation in a 7 mm diameter×10 mm deep pilot hole. When deployed, the bone anchor 100 can be low profile to prevent post-surgical irritation. The bone anchor 100 or driver 200 can comprise an indicator (e.g., etch mark) for indicating the depth of anchor 100 insertion. Tactile and/or audible feedback from the bone anchor 100 can be used to indicate anchor deployment. As the bone anchor 100 and the driver 200 can be loaded prior to use, there is a single sterile barrier such that the user is not adding or removing the bone anchor 100, the driver 200, or any components thereof during the procedure.

It should be understood that the values used above are only representative values, and other values may be in keeping with the spirit and intention of this disclosure.

While several inventive embodiments have been described and illustrated herein with reference to certain exemplary embodiments, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein (and it will be understood by one skilled in the art that various changes in detail may be effected therein without departing from the spirit and scope of the invention as defined by claims that can be supported by the written description and drawings). More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto; inventive embodiments may be practiced otherwise than as specifically described and claimed. Further, where exemplary embodiments are described with reference to a certain number of elements it will be understood that the exemplary embodiments can be practiced utilizing either less than or more than the certain number of elements.

All references, including publications, patent applications, and patents, cited herein are hereby incorporated by reference to the same extent as if each reference were individually and specifically indicated to be incorporated by reference and were set forth in its entirety herein.

All definitions, as defined and used herein, should be understood to control over dictionary definitions, definitions in documents incorporated by reference, and/or ordinary meanings of the defined terms.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. The term “connected” is to be construed as partly or wholly contained within, attached to, or joined together, even if not directly attached to where there is something intervening.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

Approximating language, as used herein throughout the specification and claims, may be applied to modify any quantitative representation that could permissibly vary without resulting in a change in the basic function to which it is related. Accordingly, a value modified by a term or terms, such as “about” and “substantially”, are not to be limited to the precise value specified. In at least some instances, the approximating language may correspond to the precision of an instrument for measuring the value. Here and throughout the specification and claims, range limitations may be combined and/or interchanged; such ranges are identified and include all the sub-ranges contained therein unless context or language indicates otherwise.

The recitation of ranges of values herein are merely intended to serve as a shorthand method of referring individually to each separate value falling within the range, unless otherwise indicated herein, and each separate value is incorporated into the specification as if it were individually recited herein.

All methods described herein can be performed in any suitable order unless otherwise indicated herein or otherwise clearly contradicted by context. The use of any and all examples, or exemplary language (e.g., “such as”) provided herein, is intended merely to better illuminate embodiments of the invention and does not impose a limitation on the scope of the invention unless otherwise claimed.

No language in the specification should be construed as indicating any non-claimed element as essential to the practice of the invention.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

It will be apparent to those skilled in the art that various modifications and variations can be made to the present invention without departing from the spirit and scope of the invention. There is no intention to limit the invention to the specific form or forms disclosed, but on the contrary, the intention is to cover all modifications, alternative constructions, and equivalents falling within the spirit and scope of the invention, as defined in the appended claims. Thus, it is intended that the present invention cover the modifications and variations of this invention provided they come within the scope of the appended claims and their equivalents.

What is claimed is:

1. A drill bit comprising:
a shaft extending to a distal end, the distal end having a drill tip;
a sharp point extending distally from the drill tip; and
a stop extending around the shaft at the drill tip, the stop having one or more cutting edges and a diameter that is greater than a diameter of the shaft.

2. The drill bit of claim 1, wherein the stop is a cylindrical collar.

3. The drill bit of claim 1, further comprising one or more recesses extending through an outer surface of the stop.

4. The drill bit of claim 3, wherein the one or more recesses extend to the one or more cutting edges.

5. The drill bit of claim 3, wherein an inner surface of the stop extends from the one or more recesses to the one or more cutting edges along an axis and the shaft extends along a central longitudinal axis that is substantially perpendicular to the axis.

6. The drill bit of claim 1, wherein an outer surface of the stop is smooth.

7. The drill bit of claim 1, wherein the drill tip comprises one or more flat faces.

8. The drill bit of claim 7, wherein two of the one or more flat faces oppose each other and converge at the sharp point.

9. The drill bit of claim 1, wherein the sharp point is substantially triangular and increases in width in a proximal direction.

10. A bone anchor comprising:
a body having at least two opposing openings forming a window extending through the body;
an expansion member having a pair of connected and spaced wings, the expansion member extending at least partially into the window in the body; and
a fixation mechanism comprising an adjustable loop, the adjustable loop extending between the pair of connected and spaced wings in the window of the body.

11. The bone anchor of claim 10, further comprising two top openings extending through a top surface of the body.

12. The bone anchor of claim 11, wherein the fixation mechanism extends through the two top openings in the top surface of the body.

13. The bone anchor of claim 10, further comprising an interface at a top surface of the body.

14. The bone anchor of claim 10, further comprising an abrasive feature on the body.

15. The bone anchor of claim 10, further comprising a projection on the expansion member extending between the pair of connected and spaced wings.

16. The bone anchor of claim 15, wherein the projection extends into the window of the body.

17. The bone anchor of claim 16, further comprising a first channel extending between one wing of the pair of connected and spaced wings and a first side of the projection.

18. The bone anchor of claim 17, further comprising a second channel extending between the other wing of the pair of connected and spaced wings and a second side of the projection.

19. The bone anchor of claim 18, wherein the adjustable loop extends within the first and second channels.

20. The bone anchor of claim 10, wherein the fixation mechanism is a length of suture having a first limb and a second limb extending from the adjustable loop.
